(19) United States
(12) Patent Application Publication
(10) Pub. No.: US 2023/0047530 A1
(43) Pub. Date: Feb. 16, 2023

(54) METHOD AND DEVICE FOR ANALYZING FINE MOTOR SKILLS

(71) Applicant: INSTITUT MINES TELECOM, PALAISEAU (FR)

(72) Inventor: Nesma HOUMANI, MELUN (FR)

(21) Appl. No.: 17/792,994

(22) PCT Filed: Jan. 13, 2021
(86) PCT No.: PCT/EP2021/050586

§ 371 Date: Jul. 14, 2022

(30) Foreign Application Priority Data
Jan. 15, 2020 (FR) FR2000383

(51) Int. Cl. G16H 50/30

(52) U.S. Cl. CPC G16H 50/30

(57) ABSTRACT

Method for acquiring and analysing the fine motor skills of an individual. Presenting a course on a medium and inviting the individual to make a free movement with at least one finger and/or one accessory on the medium, this movement being linked to the course. Recording a period of time taken to complete at least part of the course. Recording successive positions of the finger and/or the accessory during the completion of at least a part of the course. Analysing the recordings in order to generate at least one random variable describing successive positions according to a predefined statistical model. Generating a score representative of the fine motor skills, based on at least the period of time taken to complete at least part of the course and a statistical measurement of the random variable, characteristic of a quantity of information, disorder or chaos in the recording of the successive positions.

### TECHNICAL FIELD

The present invention relates to the field of data analysis in the field of cognitive and behavioral neurosciences. The invention relates more particularly to a method for analyzing fine motor skills by acquiring and analyzing the movements of an individual, and to a device for carrying out this method.

### PRIOR ART

Fine motor skills correspond to the execution of precise actions, in particular using the muscles of the fingers, and more generally of the hand, or of the face, and to the muscular control of the muscles concerned. Gripping and manipulating small objects, performing delicate gestures, such as drawing or writing, or controlling the facial muscles are examples of fine motor skills.

Methods of analyzing fine motor skills of the hand in children, available in the literature, permit identification of neurodevelopmental disorders. Indeed, the publications Handwriting performance in children with attention deficit hyperactivity disorder (ADHD), by M. Racine et al., Children with autism show specific handwriting impairments, by C. Fuentes et al., Clinical description of children with developmental coordination disorder, by L. Miller et al., Fine motor deficiencies in children with developmental coordination disorder and learning disabilities, by B. Smits-Engelsman et al., and Handwriting process and product characteristics of children diagnosed with developmental coordination disorder, by S. Rosenblum, use dysgraphia, a disorder of written expression, to identify neurodevelopmental disorders such as attention deficit with or without hyperactivity, autistic spectrum disorders, or coordination acquisition disorders. These methods are based on the analysis of handwriting in children, particularly children in elementary classes, i.e. children who know how to write. However, writing is a complex and codified action. Moreover, the process of learning to write can take more than ten years, as described in the publications Handwriting development, by K. P. Feder et al. and Factors that relate to good and poor handwriting, by H. Cornhill et al. These methods therefore do not permit early detection of neurodevelopmental disorders.

Publications also describe analyses of writing that are aimed at detecting diseases such as Parkinson’s, Huntington’s, schizophrenia, sclerosis, or various types of depression. In particular, the publications Parkinsonism reduces coordination of fingers, wrist, and arm in fine motor control, by H. L. Teulings and Spiral analysis: a new Technique for measuring Tremor with a Digitizing Tablet, by S. L. Pullman analyze tracings of Archimedean spirals, concentric circles or handwritten texts in order to detect Parkinson’s disease, in particular based on spatio-temporal parameters, durations of movements, speeds and/or fluidity of movements. However, the analyses described in the above publications are aimed at identifying a particular disease, namely Parkinson’s, and target a particular population, namely the elderly.

### DISCLOSURE OF THE INVENTION

There is therefore a need for a method permitting early identification of atypical behaviors, in particular neurodevelopmental disorders, in people of all ages, which method can be carried out easily and not just in a clinical context.

### SUMMARY OF THE INVENTION

The invention aims to meet this need and, according to one of its aspects, achieves this by virtue of a method for acquiring and analyzing the fine motor skills of an individual, comprising the following steps:
a) presenting at least one route on a support, inviting the individual to perform a free movement with at least one finger and/or an accessory on the support, this movement being linked to the route presented,
b) recording a duration of completion of at least part of the route,
c) recording the successive positions of the finger and/or of the accessory during completion of at least part of the route,
d) analyzing the recordings in order to generate at least one random variable describing the successive positions of the finger and/or of the accessory according to a predefined statistical model,
e) generating a score representative of the fine motor skills, based on at least the duration of completion of at least part of the route and a statistical measurement of the random variable, characteristic of a quantity of information, of a disorder or chaos contained in the recording of the successive positions of the finger and/or of the accessory.

Such a method is implemented using computer means. The recording is done in a computer memory, and the analysis and generation of the score are done by computer.

The invention is based on a statistical modeling of the movement performed by the individual with the finger and/or the accessory while completing the route. The statistical measurement resulting from this modeling makes it possible to identify an individual’s cognitive and motor functions, such as attention, planning and memorization, which are important markers of good health.

The invention allows the possible presence of pathologies to be detected simply and quickly, in particular by identifying an atypical development in children, an abnormal decline in the elderly, or a progression or regression during therapeutic treatment.

One of the advantages of the method according to the invention is that it allows the early detection of cognitive and/or motor disorders via the analysis of fine motor skills independently of age, of language or of the level of mastery of writing.

The route can in particular be completed without instruction and without special knowledge, making it easier for any individual to complete. Moreover, the route can be completed remotely by the individual, for example at home or at school, and does not require a trip to a healthcare specialist.

The analysis of the movement performed makes it possible in particular to identify hesitations or errors while at least part of the route is being completed.

The movement can be analyzed in particular by recording the positions of the finger and/or of the accessory during the completion of at least part of the route. The recording of speeds and/or accelerations of the movement of the finger and/or of the accessory and/or the recording of pressures of the finger and/or of the accessory on the support and/or the recording of inclinations of the finger and/or of the accessory with respect to the support during completion of at least part of the route can permit a more detailed analysis of the movement. The inclinations of the finger and/or of the accessory can be acquired by image analysis, optionally with optical markers attached to the finger and/or to the accessory (colored, reflected, light-emitting patches), and/or the accessory may possibly include an accelerometer. The acquisition of the recordings is preferably carried out with a constant sampling frequency. Preferably, the at least one part of the route corresponds to the route as a whole.

Completing several routes that may have varying degrees of difficulty makes it possible to characterize more precisely the cognitive and/or motor capacities of the individual.

The statistical modeling of these recordings makes it possible to carry out the analysis by quantifying the characteristics of the movement, such as the fluidity and/or the control of the movement, in particular via the determination of a statistical measurement.

### Statistical Modeling

The statistical measurement can be an entropy measurement, including a multi-scale entropy, an approximate entropy, a “sample entropy”, a Tsallis entropy or a differential entropy.

The entropy in fact makes it possible to measure the disorder and the uncertainty of the actions performed by the individual during the completion of the route.

The statistical measurement can also be defined from a measurement of chaos, notably a measurement of fractal dimensions or a measurement of the Lyapunov exponent.

Preferably, the statistical measurement is a differential entropy, which can be described by the following formula:

$$h \left(x\right) = - {\int }_{X} f \left(x\right) ln \left(f \left(x\right)\right) d x$$

f being the probability density of X, a random variable.

The probability density of the random variable can be estimated by a statistical model, in particular by a mixture of Gaussians (GMM). The differential entropy can then be defined by

$$h \left(t\right) = \frac{1}{2} l n \left[{\left(2 π e\right)}^{N} d e t \left(Σ\right)\right]$$

where Σ is the covariance matrix and N the dimension of the Gaussians, for example equal to 2, corresponding for example to the dimension of the coordinates of the positions of the finger and/or of the accessory. The number of Gaussians can be determined so as to limit the calculation time of the statistical measurement while retaining a quantity of information contained in the recording sufficient for the analysis. The mixture of Gaussians comprises for example 30 Gaussians or more.

The differential entropy measurement of a Gaussian is related to its variance and quantifies the dispersion of the Gaussian distribution. An uncoordinated movement will therefore have a high entropy measurement, unlike a fluid action. Variance can be defined as a statistical measurement, likewise an average or any other value relative to the statistical model.

Alternatively, the statistical model can be a hidden Markov model, characterized by a number of states S. The states can correspond to portions of completion of the route, that is to say to a position or a set of successive positions of the finger and/or of the accessory. The number of states S may depend on the length of time the individual takes to complete the route. The states may correspond to portions of completion of the route, each extending over the same duration, and/or the states can comprise the same number of successive positions of the finger and/or of the accessory. Thus, the longer the completion time, the greater the number of states S.

Alternatively, the number of states S can be predefined. The states can in particular comprise a different number of successive positions of the finger and/or of the accessory and/or can correspond to portions of completion of the route that extend over variable durations.

An intermediate statistical measurement can be calculated for each of the states S and/or an intermediate completion time can be recorded for each state of the hidden Markov model. The statistical measurement can be defined from the intermediate statistical measurements, and/or the completion time can be defined on the basis of the intermediate durations. An intermediate score representative of the fine motor skills can be generated for each state of the hidden Markov model, from at least the intermediate duration of the portion corresponding to the state and the intermediate statistical measurement calculated for the state, the score being able to be defined from the intermediate scores, the intermediate statistical measurements and/or the intermediate durations. The analysis of the fine motor skills is advantageously more refined and more precise, making it possible to locate the portion or portions of the route where the individual has shown atypical behavior.

### Calculation of the Score

The statistical measurement in combination with at least the duration of completion of the route advantageously makes it possible to generate the score representative of the fine motor skills.

However, the individual’s score may vary depending on the route followed by the individual, for example depending on the difficulty of the route, on the conditions for completing the route, on the handling of the accessory, or on the understanding of the objective of the route. Steps a), b), c), d) and e) are advantageously repeated several times for the same individual, preferably between 2 and 40 times, better between 10 and 30 times, better still about 20 times. An average score can then be calculated from the scores of each step e).

As a variant, steps a), b) and c) are repeated several times for the same individual, and steps d) and e) are carried out once on the basis of the recordings from steps b) and c) repeated several times.

Preferably, the sum of the times for carrying out step b) is less than 10 minutes. The individual’s concentration may drop when completing at least part of the route or several routes takes too long.

The analysis of several completions of the route makes it possible to obtain an overall score for the individual’s fine motor skills, characterizing more faithfully his motor and cognitive abilities.

Parameters other than the successive positions and the duration can help characterize an individual’s fine motor skills more precisely, further improving the analysis. Indeed, a movement having a constant speed during completion of the route is in particular representative of a controlled movement. Thus, a more refined analysis of the movement is for example carried out by one or more additional recording(s) of speeds and/or accelerations of the finger and/or of the accessory and/or of pressures of the finger and/or of the accessory on the support and/or of inclinations of the finger and/or of the accessory with respect to the support while completing the route, the score being generated at least from this or these recording(s).

Preferably, the analysis of the recording(s) is carried out in such a way as to generate at least a random variable describing the speed and/or a random variable describing the acceleration and/or a random variable describing the pressure of the finger and/or of the accessory and/or a random variable describing the inclination of the finger and/or of the accessory with respect to the support according to at least one predefined statistical model, the score generated in step e) being calculated from at least one statistical measurement of the at least one random variable characteristic of a quantity of information, of a disorder or chaos contained in the recording(s) of the speeds, accelerations, pressures or inclinations of the finger and/or of the accessory.

### Analysis of the Score

The identification of atypical behaviors is preferably carried out by comparing the score representative of the fine motor skills against at least one reference score. The reference score is, for example, an average score.

The score can be compared against scores recorded in a database. These recorded scores can be derived from implementations of the method according to the invention.

The comparison can in particular be carried out by calculating a z-score, then by performing a thresholding; with the z-score expressing the deviation from the mean, the thresholding then makes it possible to identify significant deviations from this mean.

The comparison can also be carried out by means of a machine learning method, in particular a classification method, for example K-means, hierarchical classification, GMM (Gaussian Mixture Model), k-medoids, DBSCAN (density-based spatial clustering of applications with noise), k-nearest neighbors, random forest, graph method. The classification methods may or may not be supervised. This list is not exhaustive.

The comparison is preferably carried out to compare scores of individuals who are largely similar, particularly in terms of age, education and/or language.

The comparison is preferably carried out for substantially similar degrees of difficulty of the route and/or for identical routes.

The comparison can also be carried out for the same individual following, for example, different routes and/or following one or more routes at different moments in time. These different moments in time can correspond to a time before treatment and a time after treatment. A change in the fine motor skills of the individual can be determined by comparing the scores resulting from the completions of the route at different moments in time.

The comparison can be carried out on the basis of intermediate scores, allowing the identification of the portions comprising an atypical behavior of the individual.

An alert can be generated according to the result of the comparison, for example with the aim of carrying out a more detailed analysis a posteriori, for example by presenting the individual with different routes, which may be of different difficulties; the alert can be communicated, for example automatically by email or SMS, by being displayed on the support and/or recorded in a database, to a specialist in cognitive and/or motor disorders, to a physician, to a person in charge of the individual, to the individual, and/or to a relative of the individual.

The generation of the score, the comparison, and/or the generation of an alert can be carried out remotely from the completion of the route and from the detection of the successive positions and/or the duration of completion of at least part of the route.
Route
The route can be randomly generated and/or saved.
The route presented can be a labyrinth.

A labyrinth preferably defines a single journey connecting a starting point to a destination point. The labyrinth can have at least one dead-end path. Preferably, the labyrinth has several dead-end paths.

It is possible in particular to select a degree of difficulty, with the number of dead-end paths in the labyrinth increasing with the degree of difficulty.

Alternatively, the route is a set of points to be connected. Preferably, the points are to be connected in a predetermined order. In particular, each point can contain information such as a number and/or a letter, defining in particular the order in which the points are to be connected. The set of points to be connected can contain a single type of information, for example only points containing numbers or only points containing letters, or different types of information, for example points containing numbers and points containing letters. The points can contain other types of information such as mathematical operations, colors, shapes. Instructions, for example audio instructions or written instructions, can if necessary be broadcast in order to guide the individual over the route.

The selection of a degree of difficulty can define the number of points to be connected and/or the type of information contained in the points to be connected and/or the number of types of different information contained in the set of points to be connected, the number of points to be connected and/or the number of types of information increasing with the degree of difficulty.

The advantage of these routes is that they can easily be implemented, both on paper and on a digital device.

The presentation of the route can comprise its display on the support. In particular, the presentation comprises the display of the route, in particular a labyrinth or a set of points, on a screen, the screen preferably being a touch screen. A tracing of the trajectory of the finger and/or of the accessory can optionally be displayed on the support. In particular, the display of the tracing tends to increase the degree of difficulty by increasing the amount of visual information to be taken in.

### Device

The invention also relates, according to another of its aspects, to a system for acquiring and analyzing fine motor skills for implementing the method according to the invention, comprising:
a support suitable for displaying the route,
a timing means for measuring a duration of completion of at least part of the route,
a detection means, detecting the positions of the finger and/or of the accessory during the completion of at least part of the route,
a memory in which the positions of the finger and/or of the accessory and/or the duration of completion of at least part of the route can be recorded,
a processing and analysis means for generating a score representative of the fine motor skills from at least the duration of completion of at least part of the route and a statistical measurement, characteristic of a quantity of information, of a disorder or chaos contained in the recording of the positions of the finger and/or of the accessory, itself generated from a random variable representative of the positions of the finger and/or of the accessory.

### Support

The support can comprise a screen, preferably comprising a touch interface making it possible to follow the movement of the finger and/or of the accessory during completion of the route. The support can be a tablet, a cell phone, or an interactive whiteboard among other possibilities.

### Accessory

The accessory used by the individual to complete at least part of the route may be a tool functioning with a tablet, for example a stylus, or any suitable writing tool such as a pencil, chalk, a pen or a felt-tip pen.

The accessory can comprise a sensor, for example a capacitive, optical, thermal, pressure or ultrasonic sensor.

### Timing Means

The timing means can be an algorithm executed by computer means, this algorithm preferably being included in the program executed by the processing and analysis means, determining the duration of completion of at least part of the route from the recordings of the successive positions of the finger and/or of the accessory. Indeed, with the processing means preferably comprising an internal clock, it is possible to determine the duration of completion of at least part of the route, knowing the sampling frequency at which the successive positions are recorded.

The timing means can alternatively be separate from the processing and analysis means, being for example a specialized electronic circuit or a program executed, during the acquisition, independently of a program executed for the processing and analysis of the recordings.

### The Timing Means Can Be Included in the Support

### Detection Means

The detection means can be a sensor, in particular a capacitive, optical, thermal, pressure or ultrasonic sensor.

The detection means can be included in the support. Alternatively, it is separate from the support.

The detection means can be integrated into the support, in particular integrated into a camera, a tablet, a cell phone, or an interactive whiteboard.

The detection means can be a camera observing the support, for example a color camera, for example of the Kinect type.

The detection means can also detect the speed and/or the acceleration and/or the pressure of the finger and/or of the accessory on the support and/or the inclination of the finger and/or of the accessory with respect to the support.

### Processing and Analysis Means

The processing means can be included in the support and/or in the detection means.

The processing and analysis means can comprise a processor. Preferably, processing algorithms make it possible to automatically generate the score from the recordings.

The processing and analysis means can comprise supervised or unsupervised learning methods performing the analysis of the score generated for the individual.

The processing and analysis means can be placed at a distance from the detection means and/or the timing means and/or the support and/or the memory.

The processing and analysis means is, for example, a computer, a tablet, a cell phone, an interactive whiteboard, a smart watch, connectable for example to a tablet, an on-board camera, or a computer program located in the cloud.

The processing and analysis of the recordings can be performed in real time or in deferred mode.

### Memory

The memory preferably comprises a database, the database possibly comprising scores representative of the fine motor skills of a set of individuals, preferably of any age, recordings of successive positions, durations of completion of at least part of at least one route, personal information relating to the set of individuals and/or to the individual, or values and/or algorithms intended for the analysis of the score.

The memory can be included in the support and/or in the processing and analysis means and/or in the detection means, being for example a RAM module, a hard disk, a USB key, an SD card or a circuit integrated for example on a processor. Preferably, access to this data is made secure, protected for example by a password.

### BRIEF DESCRIPTION OF THE DRAWINGS

The invention will be better understood from reading the following detailed description of non-limiting examples of implementation thereof, and by examining the appended drawing, in which:

FIG. 1 shows a schematic view of a device according to the invention,

FIG. 2 is a block diagram showing various elements of a device according to the invention,

FIG. 3 illustrates different steps of a method according to the invention,

FIG. 4 shows a comparison of scores representative of the fine motor skills using a classification method,

FIG. 5 shows another comparison of scores using a classification method,

FIG. 6 illustrates an example of a route while it is being completed, and

FIG. 7 shows examples of routes having different degrees of difficulty.

### DETAILED DESCRIPTION

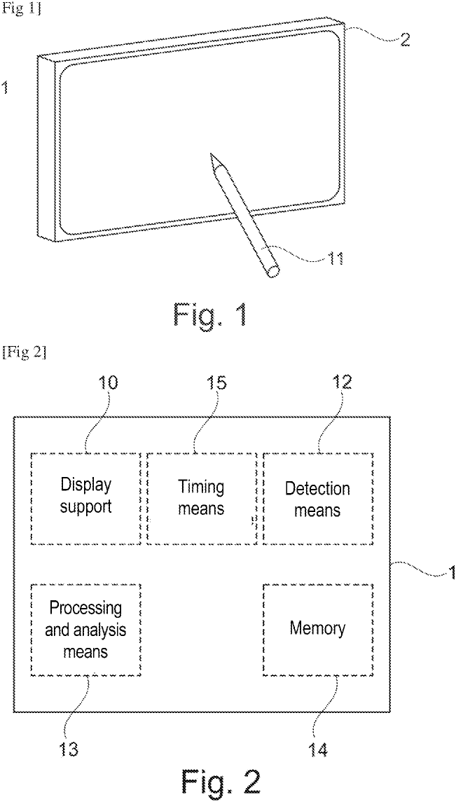

FIG. 1 shows a system 1 for acquisition and analysis of fine motor skills according to the invention, comprising a touchscreen tablet 2, for example a Wacom tablet, and an accessory 11, the accessory 11 being a stylus here. The accessory 11 comprises, for example, a tip, and an individual is able to position and move the accessory more precisely on the tablet 2.

The tablet 2 makes it possible to display a route and to detect the successive positions of the finger and/or of the accessory 11 on the screen while following at least part of the route. The accessory 11 can also help to detect the successive positions, for example by including a capacitive, optical, thermal, ultrasonic and/or pressure sensor.

The tablet 2 comprises a processing and analysis means composed in particular of a processor, analyzing the successive positions and being able to determine a duration of completion of at least part of the route, based on the acquisition of the successive positions and on an internal clock.

The tablet 2 executes a program generating and analyzing a score representative of the fine motor skills of the individual. This score can be compared against a set of scores taken from a database, it being possible for the database to be located in a memory of the tablet. The score and/or the successive positions and/or the duration of completion of at least part of the route can be recorded in the memory.

In general, the acquisition and analysis system 1 comprises a display support 10, a detection means 12, a processing and analysis means 13, a memory 14 and a timing means 15, as shown in FIG. 2.

The display support 10, the detection means 12, the processing and analysis means 13, the timing means 15 and the memory 14 may or may not be distinct from one another.

They can all be grouped together within a single appliance, for example the touchscreen tablet shown in FIG. 1.

The support 10 can alternatively be a sheet of paper, a non-touch screen, or a board.

The detection means 12 can comprise an optical, capacitive, thermal, ultrasonic and/or pressure sensor, which can be located in or under the support 10 and/or located at a distance from the support, the detection means 12 being, for example, a touch screen or a camera.

The timing means 15 can be an algorithm, preferably included in the processing and analysis means 13, determining the duration of completion of at least part of the route on the basis of the recordings of the successive positions of the finger and/or of the accessory. The processing and analysis means 13 preferably comprises an internal clock, and the duration of completion of at least part of the route can be determined on the basis of the sampling frequency at which the successive positions are recorded.

The processing and analysis means 13 can be any type of processor, in particular identical to those found in computers, tablets, cell phones, smart watches, or on-board cameras, among others.

The memory 14 can contain a database containing recorded scores and/or a set of data making it possible to carry out the processing and the analysis, for example a threshold value beyond which an alert is triggered, computer programs, personal information relating to the individual and making it possible for example to refine the analysis, such as his age, level of education, medical history. Access to this personal information is preferably made secure, for example by being protected by a password or a handwritten signature of the individual, of a healthcare professional or of any other person having a right of access to these items of personal information. The memory 14 is preferably included in the processing and analysis means 13. The processing and analysis means 13 and/or the memory 14 can be located at a distance from the support 10 and the detection means 12.

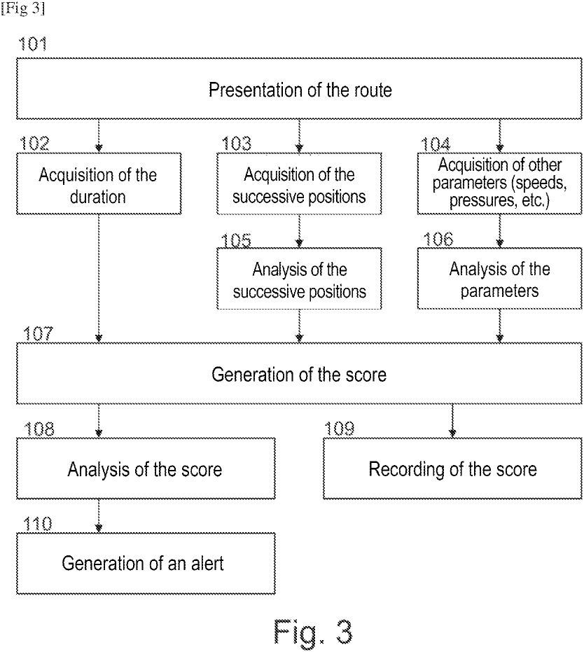

With reference to FIG. 3, different steps of a method according to the invention will now be described.

The initial step 101 involves presenting the route 26 to the individual on the support 10, and inviting the individual to perform a free movement 20 with the finger and/or the accessory on the support 10.

The duration of completion of at least part of the route and also the successive positions Mᵢ₋₁, Mi of the finger and/or of the accessory on the support 10 while completing at least part of the route are the subject of acquisitions 102, 103.

The acquisition 102 of the duration of completion of at least part of the route and/or the acquisition 103 of the successive positions of the finger and/or of the accessory on the support 10 can be carried out once the finger and/or the accessory are detected on the support 10. The acquisition 103 is preferably carried out with a constant sampling frequency. The duration of completion can be determined by computer from the number of acquisitions and the sampling frequency.

The score representative of the fine motor skills can be generated in step 107 thanks to the knowledge of the duration of completion and to the analysis 105 of the recordings of the positions comprising the generation of at least one random variable describing the successive positions of the finger and/or of the accessory according to a predefined statistical model, on the basis of which a statistical measurement is calculated. Alternatively, the successive positions of the finger and/or of the accessory can be described by a series of random variables.

In a preferred embodiment, the statistical model is a mixture of Gaussians, the statistical measurement preferably being a differential entropy. The recordings of the successive positions Mᵢ₋₁, Mi associated with the movement 20 performed by the individual on the support 10 while completing at least part of the route can be represented by a set of temporal coordinates (x(t), y(t)), the random variable consisting of at least some, better still all, of these temporal coordinates.

The random variable can also include temporal coordinates resulting from the completion of one or more routes.

A density function associated with the generated random variable can be determined from the statistical model, which can be described by the following formula in the case of a mixture of Gaussians:

$$p \left(x \left|λ\right.\right) = {\sum }_{i = 1}^{M} {w}_{i} g \left(x \left|{μ}_{i}\right. {Σ}_{i}\right)$$

with x a vector of temporal coordinates (x(t), y(t)), wᵢ weights associated with the Gaussians composing the mixture of Gaussians and

$$g \left(x \left|{μ}_{i ,}\right. {Σ}_{i}\right) = \frac{1}{{\left(2 π\right)}^{{}^{\frac{1}{2}}} {\left|{Σ}_{i}\right|}^{{}^{\frac{1}{2}}}} exp \left\{- \frac{1}{2} {\left(x - {μ}_{i}\right)}^{t} {Σ}_{i} {}^{- 1} \left(x - {μ}_{i}\right)\right\}$$

densities associated with these Gaussians, where µᵢ are means, and Σᵢ covariance matrices relating to each of the Gaussians.

The statistical measurement can be calculated using the following formula:

$$h \left(t\right) = \frac{1}{2} l n \left[{\left(2 π e\right)}^{N} d e t \left(Σ\right)\right]$$

with N = 2 being the dimension of the temporal coordinates, the statistical measurement being a differential entropy, and the statistical model a mixture of Gaussians.

Differential entropy is associated with the variance of Gaussians, quantifying the dispersion of the recordings of the successive positions. Thus, the slower the individual’s movement, for example because of reflection, hesitation, a motor problem, and/or the more the individual goes back the way, for example by having an uncoordinated gesture, the more the Gaussians are spread out, then involving a high variance and therefore a high differential entropy.

Alternatively, the statistical model can be a hidden Markov model (HMM). The hidden Markov model is notably characterized by a number of states S. The states can each be characteristic of a portion of the movement performed by the individual, a state grouping together a set of successive positions, for example, the portions preferably being distinct.

The portions can extend over substantially similar durations, the number of states S then depending on the duration of completion of at least part of the route.

Alternatively, the portions can extend over different durations, the number of states being predefined, for example.

The states can also each be characteristic of completion of at least part of the route, with the individual following several routes, for example between 2 and 40 routes, better between 10 and 30 routes, better still about 20 routes.

The states can still be characteristic of portions of the movement performed by the individual while completing several routes.

An intermediate statistical measurement is preferably calculated for each of the states S. The statistical measurement is preferably defined from intermediate statistical measurements characteristic of a state. The intermediate statistical measurements can be derived from the analysis of the portions comprising the generation of an intermediate random variable describing, for example, the successive positions included in each portion according to an intermediate statistical model, for example a mixture of Gaussians. The intermediate measurements can be differential entropies.

Intermediate scores, representative of a portion of the movement performed by the individual to complete the route, can be generated from the intermediate statistical measurements and from the durations over which said portions extend. The score can be a combination of the intermediate scores.

The score can be a weighted sum of at least the statistical measurement representative of the successive positions of the finger and/or the accessory and of the duration of completion of the route.

The score can alternatively be a vector comprising at least the statistical measurement representative of the successive positions of the finger and/or of the accessory and the duration of completion of the route.

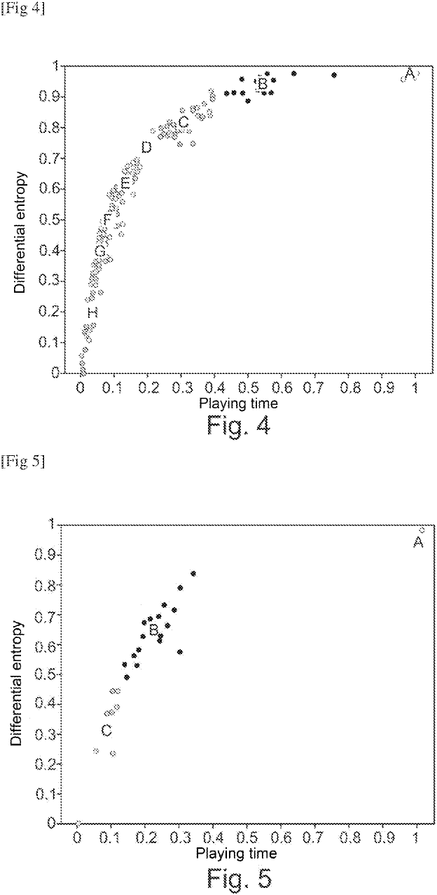

The method according to the invention comprises in particular a step 108 of analyzing the score. For example, the score can be compared automatically against scores included for example in a database. The score can be subjected to a method of classification, supervised or not, for example 2D clustering methods as shown in the examples of FIGS. 4 and 5.

Alternatively, the score is compared against a predefined threshold value, being for example an average score calculated from a set of scores resulting, for example, from the implementation of a method according to the invention. The score can be recorded in step 109. The score can enrich a database that can be used for analysis step 108 by comparison and/or participate in the definition of an average score.

The method can also include automatic generation of an alert 110 in order to warn of the detection of atypical behavior. For example, an alert can be triggered in order to draw attention to scores substantially similar to those of group A in FIGS. 4 and 5, these scores being isolated from the other groups and corresponding to a high duration of completion of the route and a high differential entropy.

FIG. 4 shows an example of comparison 108 of scores in the form of normalized vectors, the values of the duration of completion and of the statistical measurement being between 0 and 1. The comparison 108 is carried out in this example by means of a 2D clustering method, defining eight different groups, based on 258 scores of different individuals, in particular of all ages. Group A contains in particular three scores far removed from the other scores. An alert 110 can be generated concerning these three individuals, for example with the aim of carrying out new routes or medical tests.

FIG. 5 shows another example of comparison 108 of twenty-seven scores of individuals having substantially the same age, by means of a 2D clustering method on the basis of normalized vectors comprising the duration of completion of the route and the statistical measurement. In this example, the scores are classified into three groups. Group A, having a single score, may correspond to atypical behavior relating to fine motor skills.

Completion of at least part of the route can easily be done remotely and thus facilitates the implementation of the method according to the invention. Indeed, the individual can complete the route alone, that is to say without the supervision of a professional, especially a healthcare professional. The recordings and/or the score and/or the analysis can be sent a posteriori, for example by Internet, to a server which carries out the data processing.

Moreover, the method can include the step 104 of recording additional parameters, and the step 106 of analyzing these, in order to obtain a score more precisely representing the fine motor skills of the individual; the parameters can be, for example, the speeds of the finger and/or of the accessory, the accelerations of the finger and/or of the accessory, the pressures of the finger and/or of the accessory on the support 10, and/or the tremors of the finger and/or of the accessory and/or the inclinations of the finger and/or of the accessory with respect to the support while completing the route. This list is not exhaustive. These parameters can be acquired by virtue of a detection means or can be determined by virtue of a computer routine, for example determining an average or calculating a number of to and fro movements. In particular, a Wacom tablet can detect and acquire the pressures and inclinations of the accessory with respect to the tablet.

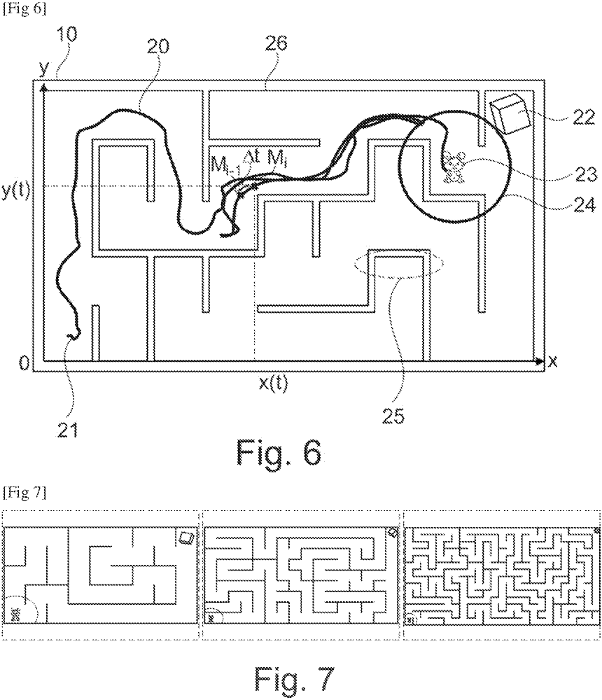

FIG. 6 illustrates an example of a route 26 that can be presented to an individual on a support, inviting him to perform a free movement 20 with his finger and/or with an accessory 11. The successive positions Mᵢ, Mᵢ₋₁ are recorded with a pitch Δt separating two successive positions and preferably constant.

A zone 24 and/or a graphic element 23 to be selected can represent the position of the finger and/or of the accessory on the support 10, making it possible in particular to visualize the position of the individual on the route. The zone 24 gives the individual a wider space, facilitating the selection for the purpose of completing the route. The zone 24 can, for example, disappear when the individual selects the zone 24 and/or the graphic element 23. The acquisition 102 of the duration and/or the acquisition 103 of the successive positions and/or the acquisition 104 of the other parameters can be performed only when the individual selects the zone 24 and/or the graphic element 23.

A tracing 20 representing the movement of the finger and/or of the accessory 11 on the support 10 can be displayed, making it possible to visualize the progress made by the individual on the route. The tracing 20 can represent the exact movement of the finger and/or of the accessory on the support 10, or it can be represented by way of segments. A tracing showing a solution of the route can also be presented to the individual at the end of the exercise, if necessary.

The route 26 can be a labyrinth, comprising at least one dead-end path 25, preferably defining a single journey connecting a starting point 21 to a destination point 22. The path can be delimited by walls, which may or may not be rectilinear, with the labyrinth taking the shape of an intestine, for example.

FIG. 7 illustrates three degrees of difficulty of the route, namely “easy”, “medium” and “difficult”, with the number of dead-end paths increasing as a function of the degree of difficulty.

The route 26 can be generated randomly, for example by means of a computer program, which is included for example in the processing and analysis means 13. This route 26 can be recorded, for example in the memory 14, and can be presented several times, for example to several individuals in order to compare their scores, and/or to the same individual, for example at different times, these different times possibly corresponding in particular to an evaluation before therapeutic treatment and to an evaluation after therapeutic treatment, in order to assess the change in the individual’s motor skills and certain cognitive functions during said treatment, and/or the effectiveness of said treatment.

Of course, the invention is not limited to the embodiments that have just been described.

In particular, the support can be a sheet of paper or any other material on which the route can be printed or drawn, the sheet or the material being positioned for example on a touch screen, or a camera being able to film the movement of the finger and/or of the accessory on the sheet of paper or the material. The movement can then be broken down in order to define successive positions, in particular by means of a computer program.

The acquisition of the successive positions can be effected by digitization of a tracing left by the individual on the support when completing at least part of the route, for example with a pen or pencil on a sheet of paper, and the duration of completion of at least part of the route can be acquired separately, for example with a stopwatch.

The steps of acquisition of the successive positions and/or of other parameters can be followed by pre-processing steps in order to standardize the recordings, typically between 0 and 1, or to identify missing recordings, for example. These pre-processing steps notably facilitate the generation and the analysis of the score.

### LIST OF CITED DOCUMENTS

Handwriting performance in children with attention deficit hyperactivity disorder (ADHD), M. Racine et al.,
Children with autism show specific handwriting impairments, C. Fuentes et al.,
Clinical description of children with developmental coordination disorder, L. Miller et al.,
Fine motor deficiencies in children with developmental coordination disorder and learning disabilities, B. Smits-Engelsman et al.,
Handwriting process and product characteristics of children diagnosed with developmental coordination disorder, S. Rosenblum,
Handwriting development, K. P. Feder et al.,
Factors that relate to good and poor handwriting, H. Cornhill et al.,
Parkinsonism reduces coordination of fingers, wrist, and arm in fine motor control, H. L. Teulings,
Spiral analysis: a new Technique for measuring Tremor with a Digitizing Tablet, S. L. Pullman.

1. A method for acquiring and analyzing the fine motor skills of an individual, comprising the following steps:
a) presenting at least one route on a support, inviting the individual to perform a free movement with at least one finger and/or an accessory on the support, this movement being linked to the route presented,
b) recording a duration of completion of at least part of the route,
c) recording the successive positions of the finger and/or of the accessory during completion of at least part of the route,
d) analyzing the recordings in order to generate at least one random variable describing the successive positions of the finger and/or of the accessory according to a predefined statistical model,
e) generating a score representative of the fine motor skills, based on at least the duration of completion of at least part of the route and a statistical measurement of the random variable, characteristic of a quantity of information, of a disorder or chaos contained in the recording of the successive positions of the finger and/or of the accessory.

2. The method as claimed in claim 1, the statistical measurement being a measurement of entropy, or a multiscale entropy, an approximate entropy, a “sample entropy”, or a Tsallis entropy, or a differential entropy or one defined from a measurement of chaos, or a measurement of fractal dimensions or a measurement of the Lyapunov exponent.

3. The method as claimed in claim 1, the predefined statistical model being a mixture of Gaussians and/or a hidden Markov model, distinguished by a number of states S.

4. The method as claimed in claim 3, wherein the predefined statistical model is a mixture of Gaussians; the mixture of Gaussians comprising 30 Gaussians or more and/orthe statistical measurement being defined by
$$h ( t ) = \frac{1}{2} ln [ {( 2 π e )}^{N} det ( Σ ) ]$$
where Σ is the covariance matrix and N the dimension of the Gaussians.

5. (canceled)

6. (canceled)

7. The method as claimed in claim 3, wherein the predefined statistical model is a hidden Markov model, distinguished by a number of states S, an intermediate statistical measurement being calculated for each of the states S.

8. The method as claimed in claim 7, the number of states S depending on a total duration of completion of the route by the individual or the number of states S being predefined.

9. (canceled)

10. The method as claimed in claim 3, wherein the predefined statistical model is a hidden Markov model, distinguished by a number of states S. comprising recording an intermediate duration of completion for each state of the hidden Markov model and/or an intermediate score representative of the fine motor skills being calculated for each state of the hidden Markov model, the score representative of the fine motor skills being able to be defined from the intermediate scores.

11. (canceled)

12. The method as claimed in claim 1, wherein
the route presented being a labyrinth or the route presented being a set of points to be connected, and/or
the route defining a single journey connecting a starting point to a destination point, and/or
the route being generated randomly.

13. The method as claimed in claim 12, wherein the route presented is a labyrinth, the labyrinth having at least one dead-end path.

14. The method as claimed in claim 13, comprising selection of a degree of difficulty, the number of dead-end paths of the labyrinth increasing with the degree of difficulty.

15. (canceled)

16. (canceled)

17. (canceled)

18. The method as claimed in claim 1, comprising the display, on the support of a tracing of the trajectory of the finger and/or of the accessory and/orthe presentation comprising the display of the route on a screen.

19. (canceled)

20. The method as claimed in claim 1, comprising the recording of speeds and/or accelerations of the movement of the finger and/or of the accessory and/or the recording of pressures of the finger and/or of the accessory on the support during completion of the route, the score being generated at least from this or these recording(s) and/or comprising the recording of inclinations of the movement of the finger and/or of the accessory with respect to the support during completion of the route, the score being generated at least from this recording.

21. (canceled)

22. The method as claimed in claim 1, steps a), b), c), d) and e) being repeated several times for the same individual.

23. The method as claimed in claim 1, steps a), b) and c) being repeated several times for the same individual, and steps e) and d) being carried out once on the basis of the recordings of steps b) and c) repeated several times.

24. The method as claimed in claim 1, comprising a step of comparing the score against scores recorded in a database.

25. The method as claimed in claim 24, comprising the generation of an alert according to the result of the comparison.

26. The method as claimed in claim 1, comprising completion of the route by the individual at different times corresponding to a time before treatment and a time after treatment; and the comparison of the representative scores of the fine motor skills resulting from completion of the route at these different times.

27. The method as claimed in claim 1, the comparison being carried out by means of a machine learning method.

28. A system for acquisition and analysis of fine motor skills for implementing the method as claimed in claim 1, comprising:
a support suitable for displaying the route, the support preferably comprising a tactile interface making it possible to follow the movement of the finger and/or of the accessory during completion of the route,
a timing means for measuring a duration of completion of at least part of the route,
a detection means detecting the positions of the finger and/or of the accessory during the completion of at least part of the route,
a memory in which the positions of the finger and/or of the accessory and/or the duration of completion of at least part of the route can be recorded,
a processing and analysis means for generating a score representative of the fine motor skills from at least the duration of completion of at least part of the route and a statistical measurement, characteristic of a quantity of information, of a disorder or chaos contained in the recording of the positions of the finger and/or of the accessory, itself generated from a random variable representative of the positions of the finger and/or of the accessory.
